(19) United States
(12) Patent Application Publication
(10) Pub. No.: US 2009/0275918 A1
(43) Pub. Date: Nov. 5, 2009

(54) PANCREATIC DELIVERY CATHETER

(75) Inventor: Michael D. CROCKER, Half Moon Bay, CA (US)

Correspondence Address: LEVINE BAGADE HAN LLP, 2400 GENG ROAD, SUITE 120, PALO ALTO, CA, 94303, US

(73) Assignee: STEMCOR SYSTEMS, INC., Menlo Park, CA (US)

(21) Appl. No.: 12/400,716

(22) Filed: Mar. 9, 2009

Related U.S. Application Data
(60) Provisional application No. 61/049,712, filed on May 1, 2008.

(51) Int. Cl. A61M 25/06; A61M 25/00

(52) U.S. Cl. 604/508; 604/525

(57) ABSTRACT

Pancreatic delivery catheters which facilitate direct intravascular access to the pancreas are described herein. A catheter which is configured particularly for pancreatic access may generally comprise a distal section which defines three curves each extending in an alternating pattern and a proximal section with an additional curve in an opposing direction. A primary curve defined along the distal section may define an acute angle with respect to a secondary curve and which also curves in an opposite direction. The secondary curve may define an acute angle with respect to both the primary curve and a tertiary curve which is proximal of the secondary curve. Similarly, a tertiary curve may extend in an opposite direction from the secondary curve. A quaternary curve along the proximal section may extend in an opposite direction from the tertiary curve.

### CROSS-REFERENCE TO RELATED APPLICATIONS

This application claims the benefit of priority to U.S. Prov. Pat. App. 61/049,712 filed May 1, 2008, which is incorporated herein by reference in its entirety.

### FIELD OF THE INVENTION

The present invention relates generally to intravascular medical devices. More particularly, the present invention relates to intravascular delivery catheters for use in accessing and delivering therapies to the pancreatic system.

### BACKGROUND OF THE INVENTION

Various types of catheters are known for a variety of applications. Many catheters are typically advanced intravascularly over a guidewire, e.g., 0.035 in, to advance a distal tip of the catheter to a region within a patient's body for treatment. During insertion and manipulation of the catheter, the catheter generally should be guided to prevent kinking and excessive bending within the vessels. Moreover, the catheter should provide sufficient stability once sufficiently positioned within the vessel despite any tortuous pathways that the catheter traverses.

However, certain regions within the body generally present an intravascular pathway which is overly tortuous to cross. Accordingly, the delivery of any medicaments or agents to these particular regions may be limited to treatments which are systemic in approach rather than targeted. An example of such a region is the pancreas, which is generally accessible intravascularly through the abdominal aorta of a patient. However, the vasculature extending from the aorta and into or through the pancreas presents a particularly tortuous pathway which is difficult to access with conventional catheter devices.

Accordingly, there is a need for an intravascular delivery catheter which is configured to access the tortuous pathways of regions within the body such as the pancreas.

### SUMMARY OF THE INVENTION

In order to intravascularly reach the pancreas, a catheter which is configured particularly for pancreatic access may be utilized. Such a catheter may generally comprise a distal section which defines three curves each extending in an alternating pattern and having an overall length of, e.g., 2 to 3 cm. A proximal section may also define a curve extending similarly. A primary curve defined along the distal section may define an acute angle with respect to a secondary curve which is proximal of the primary curve and which also curves in an opposite direction from the primary curve. The secondary curve may define an acute angle with respect to both the primary curve and the tertiary curve which is proximal of the secondary curve. The secondary curve may also curve in an opposite direction from the primary curve.

Likewise, the tertiary curve may define an acute angle with respect to the secondary curve and the proximal section. The tertiary curve may also curve in an opposite direction relative to the secondary curve and the quaternary curve, which is located along the proximal portion. The quaternary curve may define an obtuse angle along the length such that the distal portion is angled transversely with respect to the length of the proximal portion and the quaternary curve. Accordingly, each of the curves, i.e., primary, secondary, tertiary, and quaternary may curve in an alternating manner with respect to one another to facilitate placement and positioning within the tortuous pathway leading to the pancreas.

### BRIEF DESCRIPTION OF THE DRAWINGS

FIG. 1A illustrates the pancreas and the vasculature extending within and throughout the pancreas.

FIG. 1B illustrates a schematic representation of the tortuous intravascular pathway presented for direct access within the pancreas.

FIG. 2 shows a side view of a catheter configured particularly for pancreatic access.

FIGS. 3A and 3B show planar views of a pancreatic delivery catheter optionally angled relative to a plane defined by a proximal portion of the catheter.

FIGS. 4A and 4B illustrate one variation for deploying the pancreatic delivery catheter.

FIG. 5 illustrates a schematic representation of the pancreatic delivery catheter positioned within the vasculature leading to the pancreas for targeted delivery or treatment of the pancreas.

### DETAILED DESCRIPTION OF THE INVENTION

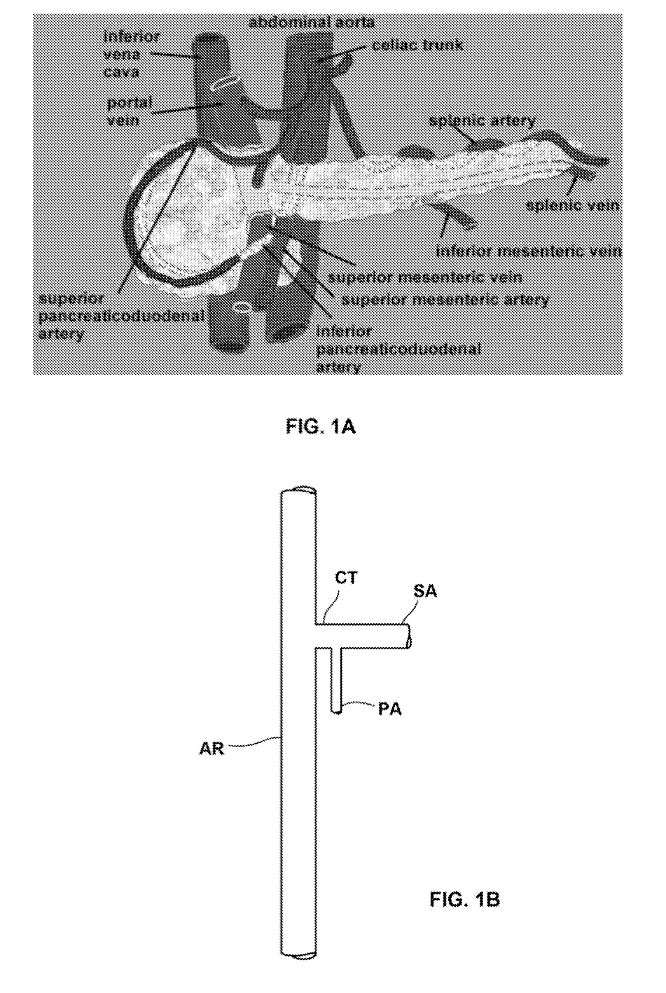

The intravascular pathways for accessing regions within the body, such as the pancreas, are particularly tortuous, as illustrated in FIG. 1A which shows the pancreas and vasculature extending within and throughout the pancreas. In order to intravascularly reach the pancreas, vessels such as the celiac trunk CT and the splenic artery SA extending from the abdominal aorta AR may be traversed. Because the celiac trunk CT extends at an acute angle from the aorta AR and the pancreatic artery PA further extends from the splenic artery SA also at an acute angle relative to the splenic artery SA, as illustrated by the schematic representation in FIG. 1B, direct intravascular catheter access to the pancreas is difficult to obtain by typical catheter devices.

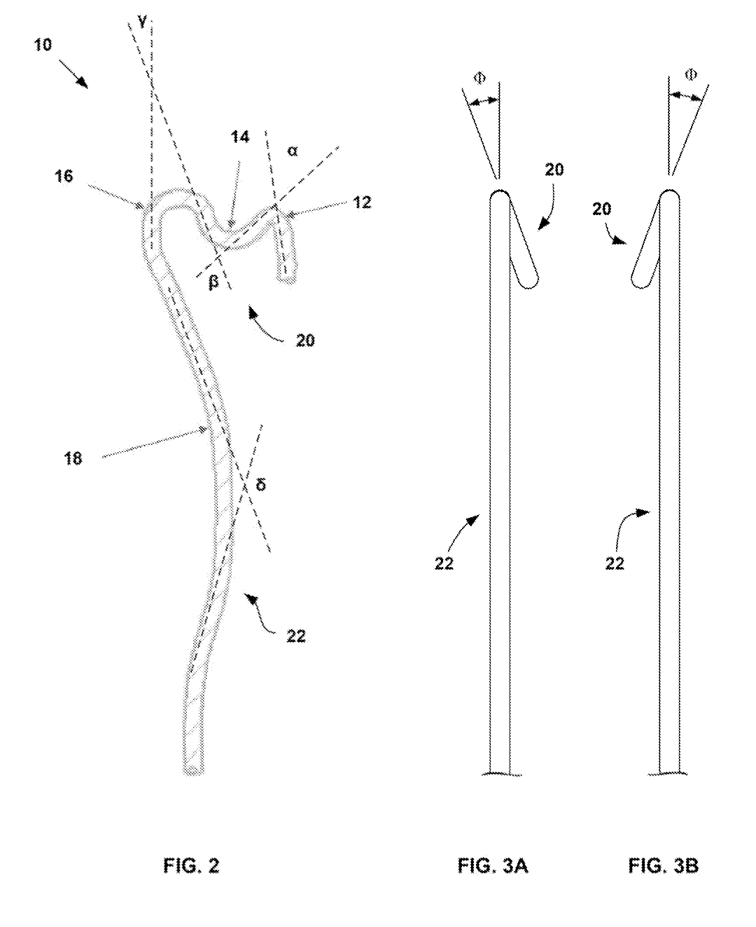

FIG. 2 shows a side view of a catheter 10 configured particularly for pancreatic access. The catheter 10 may generally comprise a distal section 20 which defines three curves each extending in an alternating pattern and having an overall length of, e.g., 2 to 3 cm. A proximal section 22 may also define a curve extending similarly. A primary curve 12 defined along the distal section 20 may define an acute angle α with respect to secondary curve 14 which is proximal of primary curve 12 and which also curves in an opposite direction from primary curve 12. Secondary curve 14 may define an acute angle β with respect to both primary curve 12 and tertiary curve 16 which is proximal of secondary curve 14. Secondary curve 14 may also curve in an opposite direction from primary curve 12.

Likewise, tertiary curve 16 may define an acute angle γ with respect to secondary curve 14 and the proximal section 22. Tertiary curve 16 may also curve in an opposite direction relative to secondary curve 14 and quaternary curve 22, which is located along the proximal portion 22. Quaternary curve 22 may define an obtuse angle δ along the length such that the distal portion 20 is angled transversely with respect to the length of proximal portion 22 and quaternary curve 22. Accordingly, each of the curves, i.e., primary 12, secondary 14, tertiary 16, and quaternary 18 may curve in an alternating manner with respect to one another to facilitate placement and positioning within the tortuous pathway leading to the pancreas.

Moreover, the distal portion 20 which may extend transversely relative to proximal portion 22 may be optionally angled relative to one another to facilitate pancreatic access. Each of the primary 12, secondary 14, and tertiary 16 curves may extend within a similar plane. This plane may be angled relative to a plane within which the proximal portion 22 lies such that an angle Φ, e.g., 20° to 30°, may be formed between the distal 20 and proximal 22 portions, as illustrated in the planar view of FIG. 3A. Alternatively, distal portion 20 may be angled in an opposing direction as illustrated in FIG. 3B. In yet other alternatives, each of the curves may be angled out-of-plane relative to one another depending upon the desired overall configuration and the anatomy of the patient to be treated.

The catheter 10 may be configured from various materials, such as polyurethane, Nylon, Pebax, etc. and may also include any number of features generally known in the art such as lengths of varying durometer whereby flexibility increases distally along the catheter, inclusion of braids or weaves, etc. Moreover, the curves along catheter 10 may be preformed such that the catheter is naturally inclined to conform to the curvatures when unconstrained. Moreover, catheter diameter may vary to typically range anywhere from 5 to 8 F or more.

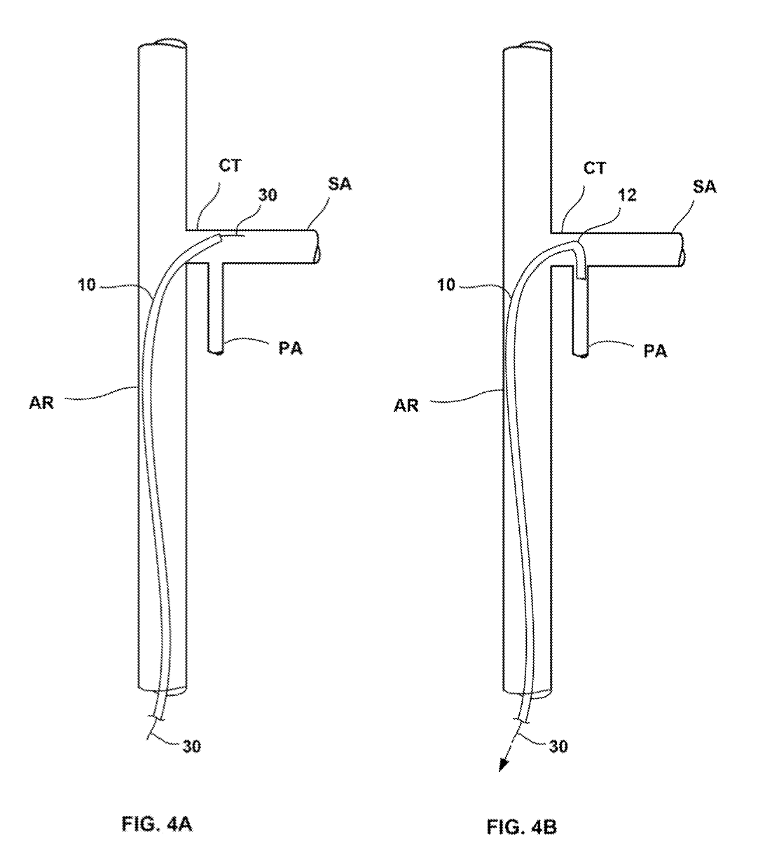

In use, with a guidewire 30 advanced through the aorta AR and within the celiac trunk CT and splenic artery SA, catheter 10 may be advanced along the guidewire 30 and until its distal end is proximate to the pancreatic artery PA, as shown in FIG. 4A. Guidewire 30 may be withdrawn proximally while maintaining a position of the catheter 10 such that the catheter may reconfigure to form each of the curves, such as primary curve 12 as the guidewire 30 is withdrawn allowing the catheter to be unconstrained and to naturally reconfigure itself. The distal end of catheter 10 may thus reconfigure itself into its curved configuration to position itself at least partially within the pancreatic artery PA, as illustrated in FIG. 4B. In this manner, guidewire 30 may be withdrawn proximally until the distal 20 and proximal 22 portions are unconstrained to reconfigure.

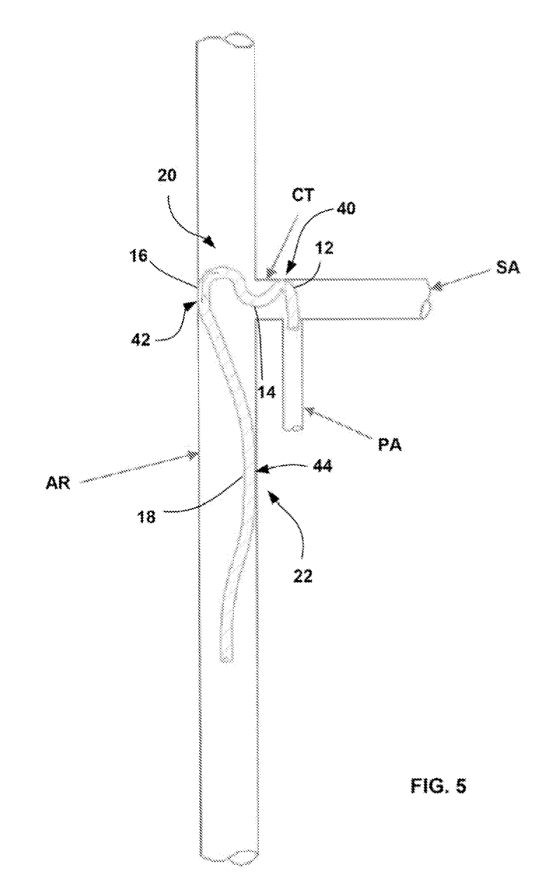

Once the guidewire 30 has been withdrawn and the catheter 10 reconfigured, the distal end of catheter 10 may be positioned at least partially within pancreatic artery PA, as illustrated in FIG. 5, to delivery drugs or agents such as stem cells for diabetes treatment or any other therapeutic medicaments directly to the pancreas. A second catheter 46 having a diameter, e.g., 2 to 5 F, may be optionally passed through the inside of the catheter 10 to sub-select a smaller artery. With the reconfigured catheter 10, primary curvature 12 may reside against the interior vessel wall 40 along the celiac trunk CT opposite to the ostium of the pancreatic artery PA to provide for stabilization of the catheter 10. Tertiary curve 16 may also reside against the vessel wall 42 of the aorta AR opposite to the ostium of the splenic artery SA and quaternary curve 18 may further reside against the vessel wall 44 along the aorta AR. These multiple points of contact between length of the catheter 10 and the vessel walls may further provide for stabilizing the catheter position with respect to the pancreatic artery PA to ensure treatment to the pancreas.

It is apparent to one skilled in the art that various changes and modifications can be made to this disclosure, and equivalents employed, without departing from the spirit and scope of the invention. Elements shown with any variation are exemplary for the specific variation and can be used on or in combination with any other variation within this disclosure.

We claim:

1. A catheter configured for pancreatic access, comprising:
a catheter having an elongate length,
wherein the length defines a primary curve defined near or at a distal end of the catheter,
wherein the length further defines a secondary curve opposite to the primary curve and proximal thereto,
wherein the length further defines a tertiary curve opposite to the secondary curve and proximal thereto, and
wherein the length further defines a quaternary curve opposite to the tertiary curve and proximal thereto.

2. The catheter of claim 1 wherein the primary, secondary, and tertiary curves are planar and comprise a distal portion of the length.

3. The catheter of claim 2 wherein the distal portion comprises a length of 2 to 3 cm.

4. The catheter of claim 2 wherein the distal portion is planar with the quaternary curve.

5. The catheter of claim 2 wherein the distal portion is angled relative to the quaternary curve.

6. The catheter of claim 2 wherein each of the curves is angled out-of-plane relative to one another.

7. The catheter of claim 1 wherein the primary curve is configured to reside against an interior vessel wall along a splenic artery opposite to an ostium of the pancreatic artery such that the distal end of the catheter is stabilized relative to the pancreatic artery.

8. The catheter of claim 7 wherein the tertiary curve is configured to reside against an interior vessel wall along an aorta opposite to an ostium of the splenic artery.

9. The catheter of claim 7 wherein the quaternary curve is configured to reside against the interior vessel wall of the aorta.

10. The catheter of claim 1 further comprising an inner catheter translatable through the catheter.

11. A method for obtaining pancreatic access, comprising:
positioning a catheter having an elongate length within a vessel lumen, the length defining a primary curve defined near or at a distal end of the catheter, a second curve opposite to the primary curve and proximal thereto, a tertiary curve opposite to the secondary curve and proximal thereto, and a quaternary curve opposite to the tertiary curve and proximal thereto,
wherein the length is stabilized with respect to the vessel lumen by one or more curves contacting against the vessel lumen.

12. The method of claim 11 wherein positioning comprises advancing the catheter intravascularly into proximity to a pancreas of a patient.

13. The method of claim 11 wherein positioning comprises positioning the primary curve against an interior of a splenic artery opposite to an ostium of a pancreatic artery such that the distal end of the catheter is stabilized relative to the pancreatic artery.

14. The method of claim 13 further comprising positioning the tertiary curve against an interior of an aorta opposite to an ostium of the splenic artery.

15. The method of claim 13 further comprising positioning the quaternary curve against the interior vessel wall of the aorta.

16. The method of claim 11 further comprising advancing an inner catheter through the catheter.

17. The method of claim 11 further comprising accessing a pancreas through the catheter.
